(19) United States
(12) Patent Application Publication
(10) Pub. No.: US 2024/0316331 A1
(43) Pub. Date: Sep. 26, 2024

(54) INFUSION SET HAVING ROLLER CLAMP

(71) Applicant: B. Braun Melsungen AG, Melsungen (DE)

(72) Inventors: Johannes Bolz, Kassel (DE); Matthias Kramer, Melsungen (DE); Claudia Freitag, Gudensberg (DE); Torben Guhl, Kassel (DE)

(21) Appl. No.: 18/578,914

(22) PCT Filed: Jul. 13, 2022
(86) PCT No.: PCT/EP2022/069646

§ 371 Date: Jan. 12, 2024

(30) Foreign Application Priority Data
Jul. 15, 2021 (DE) 10 2021 118 331.6

(51) Int. Cl. A61M 39/28

(52) U.S. Cl. CPC A61M 39/286

(57) ABSTRACT

An infusion set includes a tube and a roller clamp for regulating a flow of fluid through the tube. The roller clamp includes a housing that has side walls and a crosspiece connecting the side walls and on which the tube rests. A wheel is supported on running surfaces in or on the side walls so as to be rotatable and displaceable in the longitudinal direction of the housing. The running surfaces face the crosspiece. The lengths of the running surfaces extend in the longitudinal direction of the housing, and the widths of the running surfaces extend in the transverse direction of the housing. Movement of the wheel along the running surfaces alters a cross-section of the tube. The running surfaces each form a substantially punctiform support or contact point with the wheel, on an inner portion of the running surfaces, particularly an inner portion facing the wheel.

### CROSS-REFERENCE TO RELATED APPLICATION(S)

This application is the United States entry of International Application No. PCT/EP2022/069646, filed on Jul. 13, 2022, and claims priority to German Application No. 10 2021 118 331.6, filed on Jul. 15, 2021. The contents of International Application No. PCT/EP2022/069646 and German Application No. 10 2021 118 331.6 are incorporated by reference herein in their entireties.

### FIELD

The present disclosure relates to an infusion set having a roller clamp provided to adjust a cross-section of a tube for regulating a drip rate of a fluid which flows through the tube.

### BACKGROUND

For placing an infusion in a patient, so-called infusion sets are used. The infusion sets are made of an interface for the connection to a container (drip chamber/spike), a patient port for coupling to a patient interface (patient access), a tube for transporting/guiding a fluid, as well as a roller clamp to adjust the flow rate through the tube. A common distance of the roller clamp from the container (drip chamber) is about 25 cm. Said delivery status offers proper comfort to most users, as a fluid container to which the infusion set is connected is usually suspended upside down from a tripod and, thus, the roller clamp is arranged at a height which is conveniently accessible for an operating person. The roller clamp is delivered in the open state.

Roller clamps which restrict the flow of a fluid through the elastic tube are known from the following patent specifications.

U.S. Pat. No. 3,900,184 A discloses a roller clamp for compressing an elastic tubing. Said roller clamp includes a housing and a wheel supported in the housing to be rotatable and displaceable. The wheel compresses/presses the tube to reduce the flow of a fluid through the tube. For this purpose, a bearing surface/support surface for the tube positioned opposite to the wheel in the housing is ramp-shaped so that a play/space between the tube and the ramp becomes increasingly smaller and the tube consequently is increasingly compressed, the further the wheel is displaced along the tube/along the ramp-shaped bearing surface.

From EP 1 452 202 A1, also a roller clamp is known. Said roller clamp has a roller clamp housing and a wheel supported to be longitudinally displaceable and to be rotatable in the roller clamp housing. The wheel is cut out at its circumferential side. The roller clamp housing includes a notch matching the cut-out wheel. The roller clamp housing and the wheel jointly form a space into which the tube is pressed. The notch becomes smaller/narrower in the longitudinal direction of the roller clamp housing and in the longitudinal direction of the tube inserted in the same. Thus, the space for the tube becomes smaller and the tube is compressed more strongly, allowing the flow through the tube to be restricted.

The publications U.S. Pat. No. 8,544,815 B2, GB 1 212 781 A, U.S. Pat. Nos. 4,660,802 A, 5,352,214 A substantially have the same function as the afore-mentioned disclosures, namely to regulate the flow of a fluid through a fluid conduit, but they have structures which are different from each other.

It is a problem of the existing solutions that the adjustment path for or in the range of a lower drip rate is smaller than the adjustment path for or in the range of a higher drip rate. In other words, it was found that a longitudinal adjustment of a wheel has a higher impact on the change of the drip rate in the range of a lower drip rate than in the range of a higher drip rate. This makes it more complicated for a user to precisely adjust a low drip rate than to adjust a higher drip rate. Moreover, the forces required to adjust a low drip rate are relatively high so that it is inconvenient for the user to adjust the drip rate.

Conventional roller clamps thus are sometimes difficult to manipulate and inconvenient to adjust. Therefore, it is basically aimed at improving the user friendliness. This can be achieved, for example, by further extending the adjusting range between 0 and 60 drips/minute (range of lower drip rate). Equally, infusion sets of this species are subject to high cost pressure, because they are substantially disposables. Therefore, it is intended, at the same time, to reduce the manufacturing costs. Savings can be achieved, inter alia, by less material consumption, higher output and lower maintenance costs. Also, materials which allow recyclability are preferred. The functional unit of roller clamp can be manufactured more cheaply by optimizing the component design. At the same time, the mode of operation is optimized.

Furthermore, the wheels of conventional roller clamps are difficult to adjust and are exposed to high forces during movement/adjustment. Consequently, the wheels may be damaged or may break. This can be prevented by using solid wheels which, however, exhibit unfavorable material accumulations and require a lot of material during manufacture.

### SUMMARY

In view of this technical knowledge, the object underlying the disclosure is to improve the drawbacks of existing solutions and to provide an infusion set having a roller clamp with the least possible force applied to a wheel of the roller clamp.

According to the disclosure, this object is achieved by an infusion set having a roller clamp for adjusting the drip rate on a tube.

According to one aspect, the present disclosure relates to an infusion set having a tube and a roller clamp which is attached to the tube and by which a drip rate/a flow through the tube can be reduced. For this purpose, the roller clamp includes a housing which is approximately U-shaped or C-shaped in cross-section. In the housing, the tube rests on a crosspiece/web preferably connecting the side flanks of the U-shaped or C-shaped housing. A wheel is supported on running surfaces in or on the side walls of the housing so as to be rotatable and to be displaceable in the longitudinal direction of the housing to alter a cross-section of the tube by movement/rotation of the wheel along the running surfaces. Accordingly, the running surfaces face the crosspiece and extend along/over their length in the longitudinal direction of the housing and transversely/over their width in the transverse direction of the housing. The running surfaces each form a support or contact point with the wheel which is substantially punctiform and is formed in an inner portion of the running surfaces. Preferably, the inner portion of the running surfaces is oriented toward the wheel and thus toward the inside of the housing.

The wheel is in contact with the running surfaces in the side walls of the housing. The running surfaces extend in the longitudinal direction over the entire adjustment travel of the wheel. In the width direction, the running surfaces extend transversely to the longitudinal direction of the housing over the width of the side walls or over part of the width of the side walls. In the inner portion of the running surfaces, the support, bearing or contact point is formed between the housing and the wheel or between the housing and a wheel axle of the wheel, respectively.

The substantially punctiform support or contact point between a circular/cylindrical axle of the wheel and the (linear) running surface is punctiform only in theory. The support or contact point can actually be a surface by (minimum) deformation of the elements involved. In accordance with this disclosure, also a surface resulting from (minimum) deformation is to be understood by a punctiform support or contact point.

The clamp housing has a housing running surface in or on which the wheel runs along or is supported. The wheel equally has a wheel running surface which is in contact with the housing running surface. The wheel running surface is not in contact with the housing running surface across the entire width but only in a limited contact area. Thus, at least part of the wheel running surface is exposed. The exposure of the contact area between the wheel and the housing (bearing surface) is designed so that the contact area between the wheel running surface and the housing running surface is displaced toward the middle of the wheel. In other words, the running surface of the wheel is exposed in such a way that a contact point between the housing and the wheel is displaced in the direction of the wheel center.

The limited contact area helps reduce a possible elastic deformation in the wheel axle. The load acting on the wheel is reduced by the contact area displaced toward the middle of the wheel. For example, it is reasonable to reduce the load acting on the axle of the wheel when the wall thickness of the wheel axle is reduced by a hollow.

Preferably, the support or contact point is formed at the innermost point of the respective running surface. In this way, the distance between a center of gravity of the wheel and the support or contact point can be minimized. Thus, also the load at the outsides of the wheel and the wheel axle, respectively, can be minimized.

The running surfaces of the clamp housing are preferably inclined, specifically to taper inwards, such that the (punctiform) support or contact point is formed in the inner portion of the respective running surfaces. That is, the distance between the crosspiece and the running surface can be maximum in an outer portion and can decrease toward the middle of the housing. In this way, the support or contact point can be positioned at the innermost portion of the respective running surfaces. Hence, a load on the wheel and a possible elastic deformation of the wheel axle, respectively, can be reduced.

Preferably, the wheel has a conical wheel axle so that the (punctiform) support or contact point is formed in the inner portion of the respective running surfaces. A conical wheel axle could displace the support or contact point toward the middle of the wheel, when the running surfaces are designed to be flat on or in the side walls of the housing. Thus, the advantages of the inwardly displaced support or contact point can be exploited even with a flat housing running surface. Of course, it is also conceivable that both the wheel axle has a conical design and the running surface of the housing is inclined.

In an advantageous configuration of the disclosure which may be configured in combination with other features or as a stand-alone feature, the wheel axle of the wheel is provided to be hollow. In particular, the wheel can have trunnions formed on both sides which engage in the running surface of the clamp housing. A bore which (partially) hollows the trunnions can be arranged in one side of the trunnions.

In other words, the wheel axle of the wheel is not solid but is largely hollow. The hollow extends along the axis of rotation of the wheel from one side of the wheel axle to the other side of the wheel axle. In this way, the wall thickness of the wheel is reduced. The hollow renders the wheel fit for injection molding. The wheel axle consequently is no accumulation of material in which cavities and/or air bubbles may form. The wheel consumes less material than a wheel without the hollow, and a cycle time during injection molding is reduced.

The reduction of the load acting on the wheel and on the wheel axle, respectively, by positioning the support or contact point can be a precondition for designing the wheel axle to be hollow. This would also allow the hollow wheel axle to withstand the forces resulting from displacing the wheel.

The adjusting behavior of the roller clamp is further intended to be adapted so that the (partial) travel for the adjustment of low drip rates is increased. At the same time, the (total) travel for the adjustment from the closed state to the open state (free flow) is intended to have the same length or to be shorter than before. The behavior regarding the consistency of the drip rate should at least remain the same. The adjusting forces should be as low as possible.

In the housing, the tube can rest on a crosspiece preferably connecting the side flanks of the U-shaped or C-shaped housing with a tube bearing motion link/notch/groove extending along the housing. In/on the side walls of the housing, a wheel is supported to be rotatable and to be longitudinally displaceable and the wheel can bear against the tube and, thus, indent/press the latter against the crosspiece into/onto the tube bearing motion link. When the wheel is moved/rotated, it can roll along and off the tube, allowing the wheel to press the tube into the motion link continuously. Accordingly, the width of the motion link can constantly decrease in the longitudinal direction of the housing. That is, when the wheel is displaced/moved forward in the longitudinal direction of the housing, the wheel can continuously press the tube into the tapering motion link. In this way, a pressure on the tube within the motion link can be increased constantly and the flow through the tube can be constantly reduced.

The motion link can include a (longitudinal) section in the area of a lower tube compression (with continuously increasing tube compression) and a connected (longitudinal) section in the area of a higher tube compression (with continuously increasing tube compression). The section in the area of the higher tube compression may be longer than the section in the area of the lower tube compression.

In other words, the total adjustment travel of the wheel along the tube portion inserted in the housing/along a tube bearing surface of the housing opposite to the wheel (in which a longitudinally extending tube pinch groove/tube pinch motion link is formed) can be divided into two partial adjustment travels which are connected directly to each other along the total adjustment travel. The first partial adjustment travel can relate to an area in which the tube is increasingly (preferably continuously increasingly) compressed at a level of a higher drip rate, whereas the second partial adjustment travel may relate to an area in which the tube is increasingly (preferably continuously increasingly) compressed at a level of a lower drip rate. The second partial adjustment travel is preferably dimensioned to be longer than the first partial adjustment travel. Preferably, the gradient (change of drip rate per adjustment travel unit) in the first partial adjustment travel is larger than in the second partial adjustment travel.

Accordingly, the section (in the area) of the smaller tube compression preferably is a section with a (generally) higher drip rate and the section (in the area) of the larger tube compression is a section with a (generally) lower drip rate. The adjusting force required to move the wheel and the compress the tube and, resp., to adjust the desired drip rate through the tube may be dependent on the compression of the tube onto the bottom.

On the one hand, the adjusting force is set such that the user can easily displace the wheel and, thus, can compress the tube ergonomically. On the other hand, the adjusting force may be set such that the user has a positive feeling during displacement. That is, the required adjusting force must not be too low and the displacement of the wheel must not be too easy.

The adjustment travel, which the wheel must cover to adjust a desired drip rate, can depend on the (total) length of the motion link. Since the section of the larger tube compression is preferably longer than the section of the smaller tube compression, the adjustment travel/setting travel of the lower drip rate is preferably longer than in conventional roller clamps. Since the section of the smaller tube compression is shorter than in conventional roller clamps, it may be possible not to change the total length of the motion link as compared to conventional roller clamps so that the total travel from an open state to a closed state of the roller clamp can be the same.

In other words, the tube preferably rests on the crosspiece of the housing. Thus, the crosspiece forms a tube bearing surface. The tube is preferably compressed/pressed by the wheel which is movably supported in a groove in the side wall in the longitudinal direction of the housing. In so doing, the tube can be pressed into the motion link/longitudinal groove. The width/depth of the motion link can determine the remaining cross-section of the tube. The remaining cross-section of the tube in turn influences preferably the drip rate/the flow of a fluid flowing through the tube. That is, the geometry of the motion link preferably influences the flow rate/drip rate of the fluid in the tube. The motion link can be designed so that the motion link width/depth (continuously) decreases in the longitudinal direction of the clamp housing. The motion link can have its maximum width at the front of the clamp housing and is then tapered preferably toward the other side. When the wheel is displaced in the longitudinal direction of the clamp housing, it can therefore pinch the tube more and more into the tapering motion link and in this way can reduce the cross-section of the tube. The minimum motion link width is preferably selected such that no fluid flows through the tube and, hence, with the minimum motion link width the drip rate is preferably zero.

By reducing the motion link width, the motion link can be substantially trapezoidal. Accordingly, the reduction of the motion link width is not constant, however, but the motion link includes substantially two sections each having a constant reduction and a bending point between the two sections. The first section is the section for adjusting the high drip rate, and the second section is the section for adjusting the low drip rate. The section of the lower drip rate is larger than the section of the higher drip rate. In other words, the flank of the motion link is less steep in the section of the lower drip rate than in the section of the higher drip rate. Thus, the adjustment travel in the section of the lower drip rate is larger than the adjustment travel in the section of the high drip rate, and a low drip rate can be adjusted more precisely.

The configuration of the motion link can help increase the adjustment travel for low drip rates, but the total adjustment travel from a closed state to a completely open state must not be increased. In addition, the forces required to adjust the drip rate are preferably low due to the changed motion link geometry. In this way, more convenient and more ergonomic adjustment of the roller clamp is possible.

This aspect can offer the following advantages:
Reduction of the cost per unit by less material consumption
Improved adjusting behavior with lower manipulating force
Easier adjustment at low drip rates
Longer service life of the tool for manufacturing the roller clamp

The total adjustment travel from the closed state to the open state can be dependent on the length of the motion link. The motion link length is preferably not varied by the change of geometry as compared to known roller clamps. Thus, the total adjustment travel of the roller clamp preferably is not changed, either.

In an advantageous configuration of the disclosure, which may be configured in combination with other features or as a stand-alone feature, the minimum motion link width is provided to be equal to or smaller than one third of the largest motion link width. The ratio of the minimum to the maximum motion link width has an effect on the slope of the flank of the motion link and, consequently, on the adjustment travel of the wheel.

In an advantageous configuration of the disclosure, which may be configured in combination with other features or as a stand-alone feature, the crosspiece is provided to include an edge defined preferably by a step formed in the crosspiece and extending in the transverse direction of the housing, causing the tube to abut both on the edge and on an edge portion of a through-opening opposite to the crosspiece and thus to be clamped, when the roller clamp is locked on the tube by means of the wheel.

In other words, the tube is clamped by deflection when the roller clamp is locked on the tube. In still other words, the roller clamp is fastened to the tube by clamping the tube in/on the clamp housing. The tube is preferably clamped by deflecting the tube at an edge in the crosspiece and at a through-opening in the clamp housing. In so doing, the tube cross-section cannot be deformed or can be deformed only insignificantly. Thus, the effect of the tube deformation can be strongly reduced by a knurling of the wheel and, at the same time, the required displacing force for positioning the roller clamp on the tube can be reduced.

Preferably, the edge is formed with view towards the through-opening.

In an advantageous configuration of the disclosure, which can be configured in combination with other features or as a stand-alone feature, the through-opening is provided to be designed at the lower/downstream end of the roller clamp such that the diameter of the through-opening substantially corresponds to the diameter of the tube. In other words, the through-opening at the lower end of the roller clamp is so large that the tube does not get stuck in the through-opening, but just so small that the tube has no play in the through-opening. In this way, the tube is supported in a direction transversely to the longitudinal direction of the tube. That is, the tube cannot move perpendicularly to its longitudinal direction. The support of the tube in the through-opening, along with the edge in the crosspiece, causes the tube to be locked/fixed/fastened in the roller clamp without the tube cross-section having to be deformed by the wheel.

Preferably, the wheel is displaceable in the longitudinal direction via a predefined displacement path and the edge is positioned in a middle section of the displacement path. The displacement path of the wheel can be divided into different sections. Accordingly, the displacement path may have a start section in which the roller clamp is movable relative to the tube, the middle section in which the roller clamp is clamped on the tube but fluid can flow through the tube, and an end section in which no fluid can flow through the tube.

In an advantageous configuration of the disclosure, which can be configured in combination with other features or as a stand-alone feature, a tube holder is provided to be asymmetrical. The tube holder is a bulge on a side of the housing that is prepared and designed to receive the tube. One end of the tube, usually the end that is applied to the patient, is clamped into the tube holder. For example, the tube is clamped in the tube holder when the tube is vented before it is applied to a patient. The tube holder has a deeply notched side and a flat side. The tube can be pressed into the deeply notched side and has a tight fit there.

The tube holder geometry helps adapt the optimization of positioning and constructed space. This results in the asymmetric geometry. The geometry was optimized so that a tight fit is enabled while the tube can be easily pressed in and pulled out. The clamped end of the filled IV set cannot fall out when the system is shaken. On the other hand, it is easily possible to attach and remove the tube with one hand.

In an advantageous configuration of the disclosure, which can be configured in combination with other features or as a stand-alone feature, the clamp housing is provided to have inside an elastic rib that clamps a spike of a tube. The rib has the function of a spike shield. The spike shield is preferably realized by a 3-point clamping. In this way, the slitting in the clamp housing which is common in conventional clamp rollers can be dispensed with. A sealing surface for the slitting can also be dispensed with.

Since slitting of the clamp housing is not required, the side walls/the lower side of the clamp housing are/is continuous. In other words, in contrast to prior art, the clamp housing has no slit in the side wall/flank. The slit in the side wall constitutes the spike shield in known solutions. The spike shield is used to hold and to conceal the tube when it is connected to or disconnected from the fluid container or the patient for disposal of the tube. Since no slit has to be introduced to the clamp housing, a tool by which the clamp housing is manufactured is less strained, and there is less wear on the tool. The clamping of the tube is realized by 3-point clamping. The cross-section of the clamp housing is a semi-circle and point design. The elastic element is the rib. Consequently, a fit is tighter than in the prior art and the solution offers the advantage that the sealing surface for the slitting could be dispensed with. In other words, the spike shield for completely clamping the spike is disposed in the interior of the clamp housing. The tube is clamped inside the clamp housing.

In an advantageous configuration of the disclosure, which can be configured in combination with other features or as a stand-alone feature, a wheel lifting motion link enabling the wheel to lift off the tube bearing surface is provided to be introduced in the side wall.

In other words, the wheel lifting motion link enables the wheel to evade upwards from the tube bearing surface. When the roller clamp is opened, the wheel moves to the upper side of the roller clamp. Without the wheel lifting motion link, the wheel would follow the contour of the tube bearing surface. By pressing the wheel onto the tube, the wheel could be forced out of the clamp housing underneath a housing crosspiece. The wheel lifting motion link causes the wheel to lift off the groove when the clamp roller is opened, and thus to leave the contour of the tube bearing surface. As a consequence, the contact of the wheel with the tube and, thus, the possibility of the wheel to rest on the tube is reduced. As a result, the wheel can then no longer be forced out of the housing underneath the limiting housing crosspiece.

In an advantageous configuration of the disclosure, which can be configured in combination with other features or as a stand-alone feature, the clamp housing is provided to include in the center a step on the rear of the clamp housing. The step ensures ergonomic holding of the clamp and easy closing. Based on the size, optimum interaction is created in this way.

In an advantageous configuration of the disclosure, which can be configured in combination with other features or as a stand-alone feature, the length and the width of the clamp housing are provided to be reduced as compared to prior art. This results in saving of material and reduction of costs.

Preferably, in the side wall the clamp housing includes a groove extending in the longitudinal direction of the clamp housing. The groove is intended to reduce bending forces in a wheel introducing section of the clamp housing. When assembling the wheel in the clamp housing, the wheel introducing section can be bent open so that an axle of the wheel can come to rest inside an insertion ramp and a step. Hence, the wheel can be pressed into the clamp housing along the tube axis, while the clamp housing is (temporarily) deformed. The elongated groove can reduce the force that may be required to bend the wheel introducing section. Thus, the elongated groove can facilitate (manual) assembly. The duration and cost of assembly can be reduced in this way.

In an advantageous configuration of the disclosure, which can be configured in combination with other features or as a stand-alone feature, the volume of the clamp housing is provided to amount to less than 301 mm³. Further, the volume of the clamp roller wheel is provided to amount to less than 321 mm³.

In an advantageous configuration of the disclosure, which can be configured in combination with other features or as a stand-alone feature, the length of the clamp housing is provided to be less than 54 mm. Further, the width of the clamp housing is provided to be less than 15 mm. The height of the clamp housing is less than 24 mm.

Summing up, the following advantageous effects were achieved by the subject matter according to the disclosure:
Reduction of the material costs by adapting the geometry (shortening of the housing).
Reduction of the material costs of the roller clamp wheel by designing and adapting the material for injection molding (ABS)
Longer adjustment travel for the range of low drip rates (0-60 drips/min)
Adjustment of the clamp mechanism for the tube in the parking position (delivery status): Avoiding visible deformations on the tube. The tube is not clamped but deflected.
Simplification of the tool (more wear-resistant) by closing the recess in the spike shield.
Insertion of a wheel lifting motion link to prevent the roller clamp wheel from coming out.

### BRIEF DESCRIPTION OF THE DRAWINGS

FIG. 1 shows an isometric view of a roller clamp according to the disclosure;

FIG. 2 shows a top view onto a clamp housing of the roller clamp;

FIG. 3a shows a section view of the clamp housing across B-B in FIG. 2;

FIG. 3b shows a cutout D in FIG. 3a;

FIG. 3c shows a section view of the clamp housing across F-F in FIG. 2;

FIG. 3d shows a cutout G in FIG. 3c;

FIG. 3e shows a section view of the clamp housing across C-C in FIG. 2;

FIG. 3f shows a cutout E in FIG. 3e;

FIG. 4 shows a geometry of a motion link in a top view;

FIG. 5a shows a lateral view of the clamp housing;

FIG. 5b shows a section across the clamp housing;

FIG. 6a shows a top view onto a wheel;

FIG. 6b shows a section view of the wheel across the section A-A;

FIG. 7 shows a section across the clamp housing and the wheel;

FIG. 8 shows a locking of the roller clamp on a tube by deflecting the tube;

FIG. 9 shows a longitudinal section across the roller clamp comprising a wheel lifting motion link;

FIG. 10 shows a section across a tube holder;

FIG. 11 shows a tube holder geometry;

FIG. 12 shows the length of the tube holder;

FIG. 13 shows a section across a spike shield;

FIG. 14 shows a view of a lower side of the clamp housing;

FIG. 15 shows an isometric view of the clamp housing;

FIG. 16 shows a schematic representation of a hand which encompasses the roller clamp; and

FIG. 17 shows a lateral view of the roller clamp with dimensioning.

### DETAILED DESCRIPTION

Hereinafter, the subject matter according to the disclosure shall be described by means of the figures.

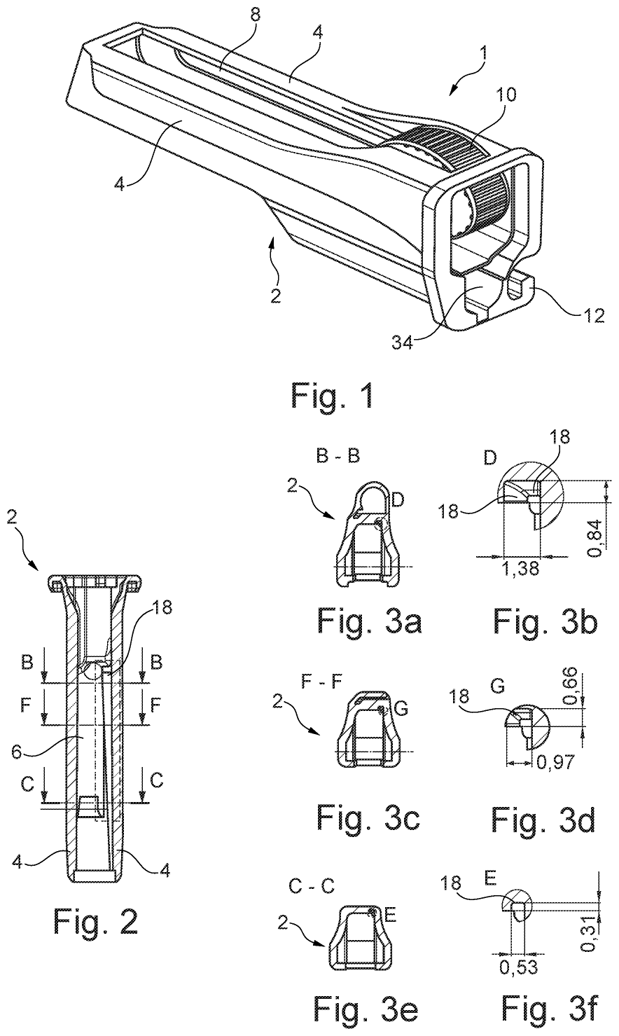

FIG. 1 illustrates a roller clamp 1 for arrangement on a tube. The roller clamp 1 includes an elongated clamp housing 2 having a substantially U-shaped or C-shaped cross-section with two opposite side walls/flanks 4 and a web/crosspiece 6 which connects the two side walls 4. A running surface 8 extends in or on each side wall, and each side wall is shaped so that such running surface 8 is created which is provided and designed to receive a wheel 10 in a rotatable and longitudinally displaceable manner. The running surface 8 extends (substantially) in parallel to the crosspiece 6 and to a tube bearing surface formed by the latter in the longitudinal direction of the clamp housing 2, respectively. The roller clamp 1 is prepared and designed to be mounted on the tube 14. To this end, the tube 14 extends on the crosspiece 6 (on the tube bearing surface). The clamp housing 2 further includes a tube holder 12 and a spike shield 34. The tube holder 12 and the spike shield 34 will be described in detail below.

The roller clamp 1 is mounted on the tube 14 such that a longitudinal end of the roller clamp 1 which is positioned closer to the wheel 10 points upwards/upstream to a drip chamber, and the other longitudinal end which is remote from the wheel 10 points downwards/downstream in the direction of a patient. Therefore, hereinafter the end having the wheel 10 will be referred to as the upper end, and the end remote from the wheel 10 will be referred to as the lower end.

FIG. 2 illustrates a top view of the clamp housing 2 without the wheel 10. The crosspiece 6 is prepared to guide the tube 14. For this purpose, the crosspiece 6 forms the tube bearing surface 20 on which the tube 14 rests. The crosspiece 6 has a motion link/longitudinal groove 18. The motion link 18 extends in the longitudinal direction of the clamp housing 2 and is offset against a side wall 4. The motion link 18 has two longitudinal ends, a front/upper end is closer to the wheel 10 and, thus, closer to the upper end of the roller clamp 1. A rear/lower end is remote from the wheel 10. From the upper end towards the lower end, the motion link 18 becomes narrower/flatter. I.e., the front end of the motion link 18 is the end having a maximum motion link width/depth, while the rear end of the motion link 18 is the end having a minimum motion link width/depth.

When the roller clamp 1 is arranged on the tube 14, that is, the tube 14 is inserted in the clamp housing 2, the tube 14 is compressed by the wheel 10. In so doing, the tube 14 is pressed onto the tube bearing surface 20 by the wheel 10. To this end, the wheel 10 is moved in the longitudinal direction of the clamp housing 2. In doing so, the tube is pressed into the motion link 18. The motion link 18 is the only free space in which the cross-section of the tube 14 can evade as soon as a compressive force is exerted on the latter by the wheel. When the motion link 18 becomes narrower/flatter in the longitudinal direction of the clamp housing 2, also the space for the tube cross-section is reduced. This means that the tube 14 is increasingly compressed by the longitudinal movement of the wheel 10 toward the lower end inside the motion link 18. As a result, the shape of the motion link 18 influences the flow through the tube 14. The motion link 18 is located laterally in the clamping section. This minimizes the influence of the tube 14 by the stress-induced flow and, where necessary, tolerances of the tube 14 are compensated in a better way.

FIGS. 3a to 3f illustrate sections across the clamp housing 2 at different positions in the longitudinal direction of the clamp housing 2. Accordingly, the cross-sections extend from a high flow rate through the tube 14 to a low flow rate through the tube 14. FIG. 3a illustrates a section across the clamp housing 2 in the section B-B in FIG. 2, viz. close to the upper end. FIG. 3b illustrates the cutout D from FIG. 3a. The cutout D shows that the motion link 18 is large at this position. Preferably, the motion link 18 has a width of 1.38 mm and a height of 0.84 mm. FIG. 3c illustrates a cross-section in the section F-F of FIG. 2. The cutout G in FIG. 3d illustrates that the motion link 18 at this height of the clamp housing 2 is smaller than in the cutout D. In this case, the motion link width preferably is 0.97 mm and the height of the motion link amounts to 0.66 mm. FIG. 3e shows a section across the clamp housing 2 in the section C-C. FIG. 3f shows a cutout E from FIG. 3e and illustrates that the motion link 18 at this height of the clamp housing 2 again is smaller. The motion link width preferably is only 0.53 mm, and the height of the motion link 18 is 0.31 mm. FIGS. 3a to 3f illustrate that the cross-section of the motion link decreases toward the lower end.

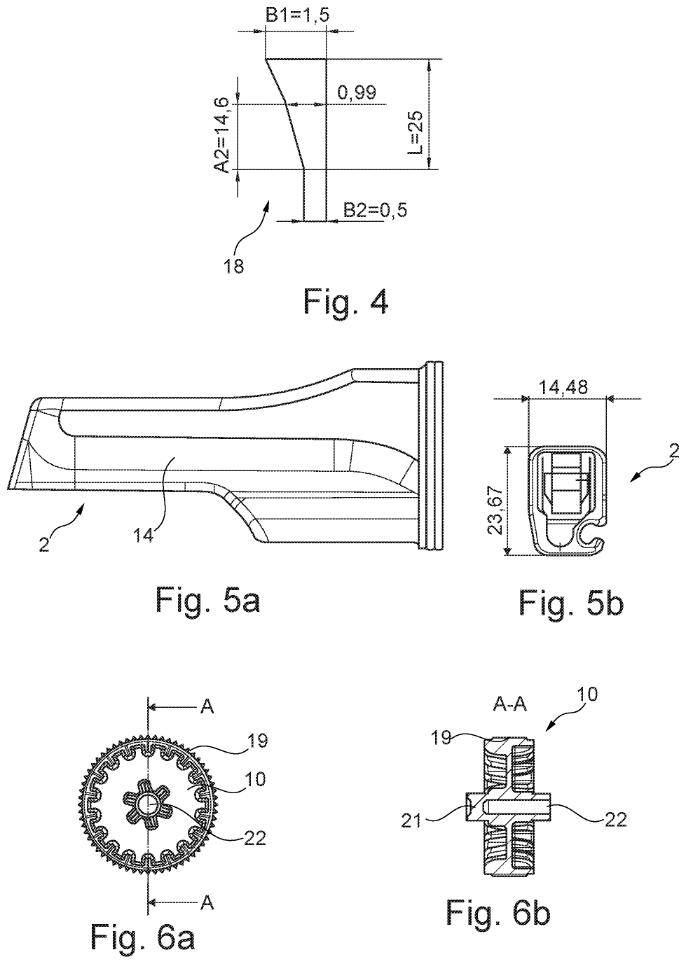

FIG. 4 illustrates the motion link geometry including dimensioning. The motion link 18 substantially takes the shape of a right-angled trapezoid. Here L is the length of the motion link 18, B1 is the maximum motion link width and B2 is the minimum motion link width. B1 is the setting for the maximum drip rate, and B2 is the setting for the minimum drip rate. Preferably, B1 amounts to 1.5 mm, B2 amounts to 0.5 mm and L amounts to 25 mm. The minimum drip rate is zero, i.e., no more fluid flows through the tube 14. The flank of the trapezoid facing the right angle has no constant inclination, however. The flank is divided into two sections, one section of a lower drip rate A2 and one section of a higher drip rate A1. The section with the higher drip rate A1 is the front section which is closer to the maximum motion link width B1 and has a larger motion link width. The section of the lower drip rate A2 is the rear section which is closer to the minimum motion link width B2 and has the smaller motion link width. The bending point K is located between the two sections A1, A2. The bending point K is defined as the point that is located between the sections A1, A2. The motion link width in the bending point is the average of B1 and B2. Preferably, the section of the low drip rate A2 has a length of 14.6 mm and the motion link width in the bending point amounts to 0.99 mm.

It is clear from FIG. 4 that the section of the lower drip rate A2 is longer than the section of the higher drip rate A1. A2 is larger than A1. Since the adjustment travel of the drip rate depends on the length of the motion link, the adjustment travel of the lower drip rate is thus larger than the adjustment travel of the higher drip rate. This allows the user to adjust a lower drip rate more precisely. Due to the less steep flank in the section of the lower drip rate, the adjusting force for the lower drip rate is lower than the adjusting force for the higher drip rate.

The area in which the fine adjustment takes place is extended appropriately. The area of the smaller cross-sectional width preferably has a length of 14.6 mm. The accompanying change of cross-section in the area before and after the bending point results in an adapted adjusting behavior. Further displacement of the bending point results, in turn, in a shortening of the adjusting range to 60 drips/min. The flow is determined by the fluid passage formed in the tube. The length of the fluid passage is determined by the compression of the wheel onto the tube.

In the case of the wheel diameter used (approx. 14.2 mm) and the predetermined compression, the wheel contact (wheel-tube) occurs over approx. 5 mm (Hertzian compression). Tests have shown that the wheel 10 must not become too small (>14.00 mm), because otherwise the initiated flow of the tube 14 is generated in a too punctiform manner (no tight closing of the fluid passage). A further increase in the wheel 10 increases the forces to be absorbed in the system and, therefore, should not exceed 15.00 mm. Otherwise, the wall thickness must be further adapted to absorb the increased forces. Also, an increase in the wheel diameter results in an increase in the roller clamp 1. The protrusion of 4 mm (+/−1 mm) of the wheel 10 from the housing caused by said situation allows good operability in the range of the maximum forces. Furthermore, there must be a lateral flow restriction of the tube for reaching the adjusting behavior (area with increased compression as compared to the main compression area).

The width of a groove of the running surface 8 must no become smaller than 0.3 mm. The groove should be designed approximately right-angled in cross-section)(90°+/−10°. A trapezoidal cross-section is also admissible.

FIG. 5a illustrates a lateral view of the clamp housing 2. The extension of the tube 14 inside the clamp housing 2 is indicated. The tube 14 is inserted into the clamp housing 2 at the front/upper end and leaves the clamp housing 2 at the lower end. Preferably, the clamp housing 2 has a length of 53.65 mm. FIG. 5b illustrates a section across the clamp housing 2 with dimensioning. The clamp housing 2 preferably has a height of 23.67 mm and a width of 14.48 mm.

FIG. 6a shows the wheel 10 of the roller clamp 1 in a top view. The wheel 10 is preferably made of plastic by injection molding, for example. The wheel 10 has a knurling 19 on the circumferential surface and is therefore nonslip. FIG. 6b shows a section view of the wheel 10 across the section A-A in FIG. 6a. The wheel 10 has trunnions 21 formed on both sides in the center which engage in the running surface 8 in the side walls 4. A bore 22 extending from one side through the center of the wheel 10 is arranged in one side of the trunnion 21. The bore 22 extends along an axis of rotation of the wheel 10.

The wheel 10 must be provided with crosspieces/ribs/the knurling 19 on the contact surface with the tube 14, because otherwise the flow of the tube is not sufficiently initiated. Otherwise, tight closing is not possible. A smooth wheel 10 is not suitable.

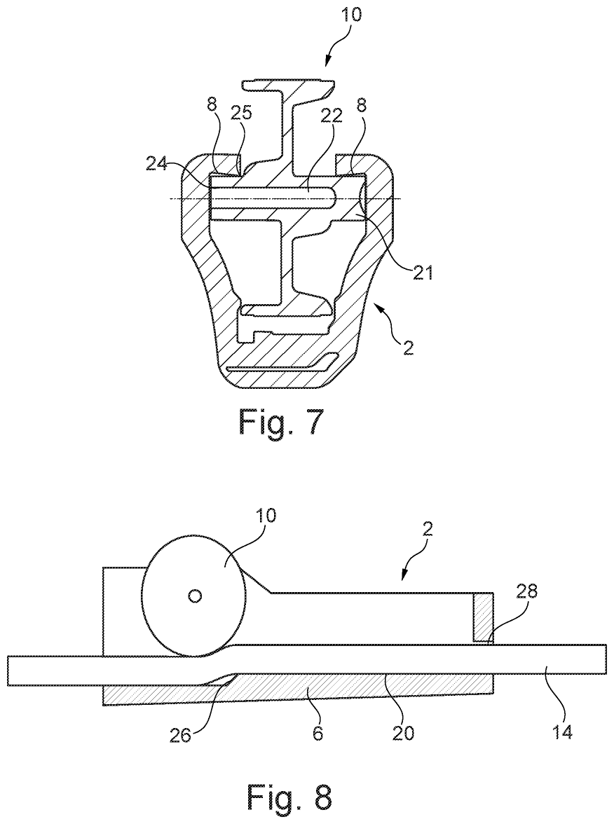

FIG. 7 illustrates a section across the clamp housing 2 at the position where the wheel 10 rests on the running surface 8 of the clamp housing 2. Accordingly, it is obvious that a clearance is provided between the clamp housing 2 and the running surface 8 and/or the running surface 24 of the wheel 10, respectively. This means that the running surface 8 and the wheel are not in contact with each other over the whole length of the running surface 8 but only in a substantially punctiform support, bearing or contact point 25. In order to realize the clearance, the running surface 8 is inclined, for example. The support or contact point 25 or the contact area between the running surface 8 of the clamp housing 2 and the wheel 10 is displaced by the inclination of the running surface 8 in the direction of a wheel center and, resp., the housing interior. This results in the substantially punctiform support or contact point 25 between the running surface 8 of the clamp housing 2 and the running surface 24 of the wheel 10. By displacing the support or contact point 25 toward the wheel center, the forces during movement of the wheel 10 which act on the wheel 10 and on the trunnions 21 of the wheel 10, resp., are reduced.

Since the mechanical properties of the wheel 10 partially were somewhat deteriorated by optimizing the injection molding, the clamp housing 2 has been adapted. The adaptation relates to the design of the contact surface between the running surface 24 of the wheel 10 and the clamp housing 2. The contact area is displaced maximally toward the wheel center by a clearance of the running surface 24. As a result, the possible elastic deformation in the wheel axle is reduced and the lower rigidity of the wheel axle due to the hollow/bore 22 is thus counteracted.

The drip rate consistency is a complex interaction of the deformations of the clamp housing 2, the wheel 10 and the tube 14. The elasticity in the system must be kept as low as possible compared to the tube so as to create a highly constant drip rate. The achieved improvement of the rigidity of the entire system of the roller clamp 1 also serves for achieving the highest possible drip rate consistency.

FIG. 8 illustrates a schematic view of locking the roller clamp 1 on the tube 14. In order to fix the roller clamp 1 on the tube 14, for example in the delivery status of the system, the roller clamp 1 is clamped onto the tube 14. To this end, the tube 14 is deflected in the roller clamp 1. The first deflection is realized by a step 26 in the crosspiece 6. The tube 14 would be pressed upwards by the step 26 and would leave the contour of the tube bearing surface 20. This is not possible through the through-opening 28 of the clamp housing 2, however. The diameter of the through-opening 28 is only insignificantly larger than the tube diameter. As a result, the tube 14 can move back and forth freely in its longitudinal direction, but it cannot move transversely to its longitudinal direction. Therefore, the step 26 and the through-opening 28 together form a deflection which fixes/locks the tube 14 in the clamp housing 2.

Previously, the roller clamp 1 was locked on the tube 14 by deforming the tube cross-section by the wheel 10. As a consequence, a deformation occurred on the tube 14 by the knurling 19 of the wheel 10. The concept was modified to the effect that the tube 14 is substantially deflected and is no longer deformed in its cross-section. For clamping by deflection an appropriate counter bearing is required, however. Therefore, the through-opening 28 was minimized at the lower end of the roller clamp 1 (on the right). By adapting the concept, the effect of the tube deformation by the knurling 19 was strongly reduced and, at the same time, the displacing force required for positioning the roller clamp 1 on the tube 14 was reduced.

FIG. 9 illustrates a wheel lifting motion link 30 in the side wall 4. The wheel lifting motion link 30 enables the wheel 10 to lift off the tube bearing surface 20. Without the wheel lifting motion link 30, the wheel 10 would follow the tube bearing surface 20 when the roller clamp 1 is opened. By pressing the wheel 10 onto the tube 14, the wheel 10 is enabled to be forced out of the clamp housing 2 underneath a housing crosspiece 32 of the clamp housing 2. The wheel lifting motion link 30 minimizes the pressing of the wheel 10 onto the tube 14 by allowing the wheel 10 to leave the contour of the tube bearing surface 20 upwards in the direction of the arrow in FIG. 9.

The wheel lifting motion link 30 is introduced in the side wall 4. The wheel lifting motion link 30 prevents the roller clamp wheel 10 from following the contour of the tube bearing surface 20 when the roller clamp 1 is opened. As a consequence, this reduces the contact with the tube 14 and, thus, the possibility of the wheel 10 resting on the tube 14.

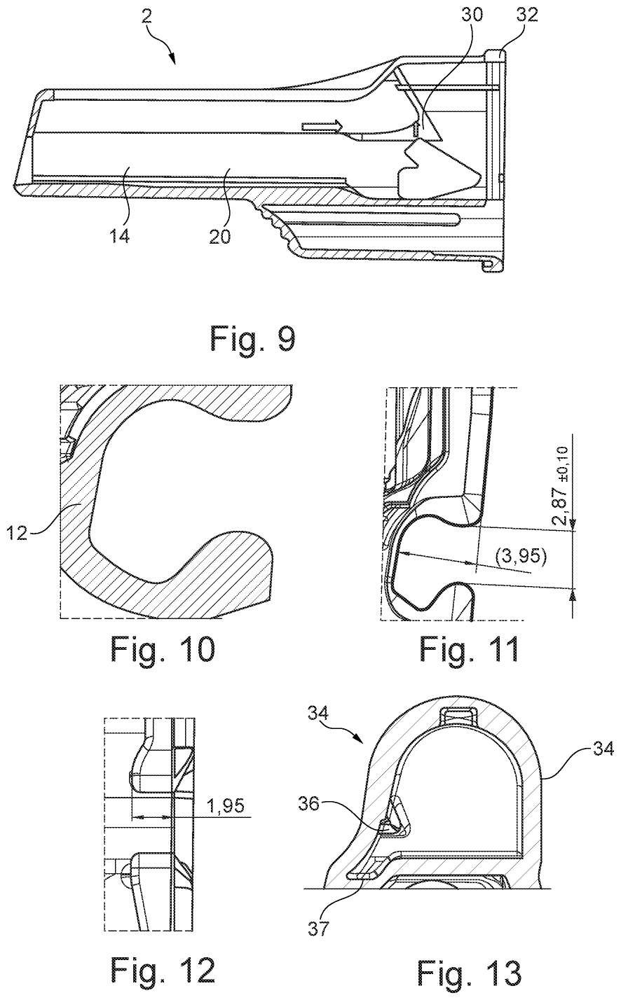

FIG. 10 illustrates a side view of the tube holder 12. The tube holder 12 is asymmetrical. The tube holder 12 is a projection from one side of the clamp housing 2 and has a substantially semicircular design. The tube holder 12 has a deeper indentation on the lower side into which the tube is tightly pressed. The upper side of the tube holder 12 is not indented as deeply as the lower side. As a result, the tube holder 12 is asymmetrical. The geometry was optimized so that a tight fit is enabled when the tube 14 is simply pressed in and pulled out. In order to obtain this behavior, the opening must be in a range of 2.9+/−0.15 mm. The width and the circumference, resp, must be adapted to the diameter of the tube 14. FIG. 11 shows the tube holder geometry. For a tube 14 having a nominal diameter of 4.1 mm, the circumference amounts to 13.9+/−1.5 mm. The length of the holder must be at least 1.8 mm (1.95+1.0/−0.15). The geometry on the upper side (on the right) was optimized regarding the tool concept and a possible damage of the packaging. FIG. 12 illustrates that the length of the tube holder 12 amounts to 1.95 mm.

FIG. 13 illustrates a section across the so-called spike shield 34 as mentioned in the foregoing already. The spike shield 34 is made substantially of a semicircle/U-profile 35 which is integrally connected to the lower side of the crosspiece 6 with its free longitudinal edges so as to form a closed cross-sectional profile. The semicircle/U-profile 35 includes an inner elastic rib 36 close to the widest point/remote from the apex of the U-profile close to one of the free longitudinal edges. The rib 36 is formed at an inner bulge 37 of the spike shield 34 which extends in the direction of the clamp housing 2. The bulge is incorporated in the crosspiece 6. A spike at the end of the tube 14 can be inserted into the spike shield 34 in the longitudinal direction of the housing 2 and is clamped by the rib 36 so that the spike is retained in the spike shield 34. The spike shield 34 is used, for example, when the tube 14 is removed from the patient. When disposing of the infusion set, the spike of the tube is inserted/clamped into the spike shield 34 so that the user does not get injured by the spike.

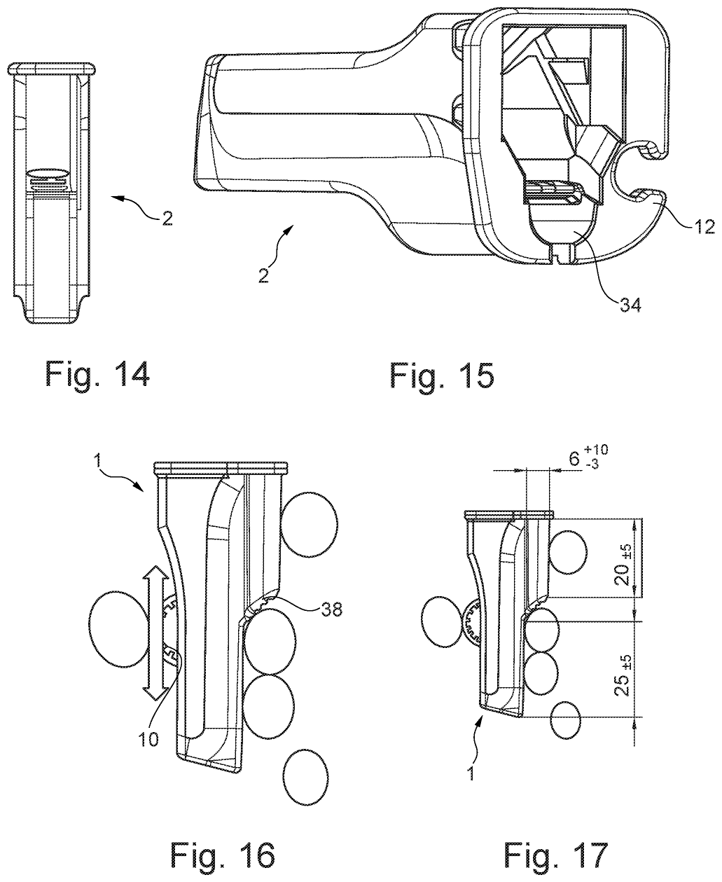

In prior art, the spike shield was realized by a slitting in the lower side of the housing. The production of the slit wears the tool by which the clamp housing is manufactured. In addition, a sealing surface for the slitting must be provided. The spike shield 34 according to the disclosure, however, is stronger than the conventional spike shield. FIG. 14 shows the lower side of the clamp housing 2. It is visible that there is no slitting in the clamp housing 2.

FIG. 15 illustrates an isometric representation of the upper end of the clamp housing 2. There is also depicted the spike shield 34.

FIG. 16 illustrates a schematic view of a hand (five symbolized finger tips) which encompasses the roller clamp 1. Accordingly, a user grasps the roller clamp 1 in such a way that the user can adjust the roller clamp wheel 10 with his/her thumb. The clamp housing 2 includes a step 38 at approximately half the height/length of the clamp housing 2. Said step 38 is designed so that the user can hold the roller clamp 1 ergonomically in his/her hand. The step 38 at approximately the middle of the rear of the roller clamp ensures ergonomic holding of the roller clamp 1 and easy closing. Based on the size, this creates optimum interaction.

FIG. 17 illustrates a lateral view of the roller clamp 1 with dimensioning. It is clear that the upper part of the roller clamp 1 up to the step has a length of 20 mm. The lower part from the step to the lower end has a length of 25 mm. Each of the two dimensions has a tolerance of +/−0.5 mm. The step has a height of 6 mm. The tolerance of the step preferably is 0.6 mm upwards and 0.3 mm downwards.

1.-15. (canceled)

16. An infusion set comprising:
a tube; and
a roller clamp,
the roller clamp configured to regulate a flow of fluid through the tube,
the roller clamp comprising a housing and a wheel,
the housing being U-shaped or C-shaped in cross-section and comprising side walls and a crosspiece that connects the side walls,
the tube resting on the crosspiece,
the side walls comprising running surfaces in or on the side walls,
the running surfaces facing the crosspiece,
each running surface comprising a length and a width,
the widths of the running surfaces extending in a transverse direction of the housing and the lengths of the running surfaces extending in a longitudinal direction of the housing,
the wheel comprising a wheel axle extending along an axis of rotation of the wheel,
the wheel being supported on the running surfaces so as to be rotatable and displaceable in the longitudinal direction of the housing,
the wheel and the tube arranged such that a movement of the wheel along the running surfaces alters a cross-section of the tube,
the running surfaces being inclined with respect to the axis of rotation of the wheel,
the running surfaces and the wheel axle forming a support or contact point that is substantially punctiform,
the support or contact point being positioned on an inner portion of the wheel axle, and
the inner portion of the wheel axle facing a middle section of the wheel when viewed in a width direction.

17. The infusion set according to claim 16, wherein the wheel axle is formed conically and wherein the support or contact point is formed in an inner portion of the running surface.

18. The infusion set according to claim 16, wherein the wheel axle is hollow.

19. The infusion set according to claim 18, wherein the wheel comprises trunnions extending to both sides of the wheel,
wherein the wheel axle comprises a bore extending along the axis of rotation of the wheel, and
wherein the bore is provided at least in one of the trunnions.

20. The infusion set according to claim 16, wherein the support or contact point is formed on a maximally innermost portion of the running surfaces, when viewed in the width direction.

21. The infusion set according to claim 16, wherein the crosspiece comprises a motion link,
wherein the motion link becomes narrower and/or flatter in the longitudinal direction of the housing,
wherein the wheel presses the tube into the motion link during longitudinal movement of the wheel,
wherein pressing of the wheel onto the tube increases during longitudinal movement of the wheel and a cross-sectional area of the tube decreases.

22. The infusion set according to claim 21, wherein the motion link comprises a longitudinal section of a generally smaller tube pressing and a longitudinal section of a generally larger tube pressing, and
wherein the longitudinal section of the generally larger tube pressing is longer than the longitudinal section of the generally smaller tube pressing.

23. The infusion set according to claim 22, wherein a width of the longitudinal section of the generally smaller tube pressing motion link of the smallest tube pressing is larger than a width of the section of the largest tube pressing.

24. The infusion set according to claim 16, wherein the crosspiece comprises an edge,
wherein the housing comprises a through-opening,
wherein the through-opening comprises an edge portion facing the crosspiece, and
wherein the tube abuts both on the edge portion of the through-opening and the edge of the crosspiece when the roller clamp is locked on the tube by means of the wheel.

25. The infusion set according to claim 24, wherein the edge is defined by a step formed in the crosspiece and wherein the step extends in the transverse direction of the housing.

26. The infusion set according to claim 24, wherein the diameter of the through-opening of the clamp housing corresponds substantially to the diameter of the tube.

27. The infusion set according to claim 24, wherein the wheel is displaceable in the longitudinal direction over a pre-defined displacement path and wherein the edge is positioned in a central portion of the displacement path.

28. The infusion set according to claim 16, wherein the clamp housing comprises an asymmetrical tube holder.

29. The infusion set according to claim 16, wherein the clamp housing comprises a rib inside the clamp housing configured to clamp a spike of the tube.

30. The infusion set according to claim 16, wherein the side wall comprises a wheel lifting motion link,
wherein the crosspiece forms a tube bearing surface on which the tube rests, and
wherein the wheel lifting motion link enables the wheel to lift off the tube bearing surface.
